SEQUENCE LISTING (21 sequences)

SEQ ID NO: 1 - protein, 75 aa, Artificial Sequence
  source: /note=“Description of Artificial Sequence Synthetic polypeptide”
  MOD_RES (1)..(1): Any amino acid
  MOD_RES (5)..(5): Any amino acid
  MOD_RES (8)..(9): Any amino acid
  MOD_RES (12)..(12): Any amino acid
  MOD_RES (16)..(25): Any amino acid
  SITE (16)..(25): /note=“This region may encompass 1-10 residues”
  MOD_RES (28)..(28): Any amino acid
  MOD_RES (32)..(32): Any amino acid
  VARIANT (34)..(34): /replace=“K” or “R”
  MOD_RES (35)..(36): Any amino acid
  MOD_RES (39)..(39): Any amino acid
  VARIANT (43)..(43): /replace=“K” or “R”
  SITE (1)..(75): /note=“Variant residues given in the sequence have no preference with respect to those in the annotations for variant positions”
  SITE (44)..(75): /note=“This region may encompass one of the following sequences ′VD′ or ′VDGQAGQGGGSGLNDIFEAQKIEWHEHHHHHH′ or ′GQAGQGGGSGLNDIFEA QKIEWHEHHHHHH′ or ′VDGLNDIFEAQKIEWHEHHHHHH′ or ′GLNDIFEAQKIEWHEH HHHHH′ or it may be absent”
  source: /note=“See specification as filed for detailed description of substitutions and preferred embodiments”
XQRRXFIXXLRXDPSXXXXXXXXXXSAXLLAXAAXXNDXQAPAXXXXXXXXXXXXXXXXXXXXXXXXXXXXXXXX

SEQ ID NO: 2 - protein, 24 aa, Artificial Sequence
  source: /note=“Description of Artificial Sequence Synthetic peptide”
VDAKFDKELEEARAEIERLPNLTE

SEQ ID NO: 3 - protein, 24 aa, Artificial Sequence
  source: /note=“Description of Artificial Sequence Synthetic peptide”
VDAKFDKELEEARAKIERLPNLTE

SEQ ID NO: 4 - protein, 24 aa, Artificial Sequence
  source: /note=“Description of Artificial Sequence Synthetic peptide”
VDAKFDKELEEVRAEIERLPNLTE

SEQ ID NO: 5 - protein, 24 aa, Artificial Sequence
  source: /note=“Description of Artificial Sequence Synthetic peptide”
VDAKFEKELEEARAEIERLPNLTE

SEQ ID NO: 6 - protein, 24 aa, Artificial Sequence
  source: /note=“Description of Artificial Sequence Synthetic peptide”
VDAKFDKELEEIRAEIERLPNLTE

SEQ ID NO: 7 - protein, 24 aa, Artificial Sequence
  source: /note=“Description of Artificial Sequence Synthetic peptide”
VDAKFDKELEEARAEIERLPALTE

SEQ ID NO: 8 - protein, 5 aa, Artificial Sequence
  source: /note=“Description of Artificial Sequence Synthetic peptide”
MAQGT

SEQ ID NO: 9 - protein, 32 aa, Artificial Sequence
  source: /note=“Description of Artificial Sequence Synthetic polypeptide”
VDGQAGQGGGSGLNDIFEAQKIEWHEHHHHHH

SEQ ID NO: 10 - protein, 30 aa, Artificial Sequence
  source: /note=“Description of Artificial Sequence Synthetic polypeptide”
GQAGQGGGSGLNDIFEAQKIEWHEHHHHHH

SEQ ID NO: 11 - protein, 23 aa, Artificial Sequence
  source: /note=“Description of Artificial Sequence Synthetic peptide”
VDGLNDIFEAQKIEWHEHHHHHH

SEQ ID NO: 12 - protein, 21 aa, Artificial Sequence
  source: /note=“Description of Artificial Sequence Synthetic peptide”
GLNDIFEAQKIEWHEHHHHHH

SEQ ID NO: 13 - protein, 67 aa, Artificial Sequence
  source: /note=“Description of Artificial Sequence Synthetic polypeptide”
  MOD_RES (25)..(25): Any amino acid
  MOD_RES (29)..(29): Any amino acid
  MOD_RES (32)..(33): Any amino acid
  MOD_RES (36)..(36): Any amino acid
  MOD_RES (40)..(49): Any amino acid
  SITE (40)..(49): /note=“This region may encompass 1-10 residues”
  MOD_RES (52)..(52): Any amino acid
  MOD_RES (56)..(56): Any amino acid
  VARIANT (58)..(58): /replace=“K” or “R”
  MOD_RES (59)..(60): Any amino acid
  MOD_RES (63)..(63): Any amino acid
  VARIANT (67)..(67): /replace=“K” or “R”
  SITE (1)..(67): /note=“Variant residues given in the sequence have no preference with respect to those in the annotations for variant positions”
  source: /note=“See specification as filed for detailed description of substitutions and preferred embodiments”
VDAKFDKELEEARAEIERLPNLTEXQRRXFIXXLRXDPSXXXXXXXXXXSAXLLAXAAXXNDXQAPA

SEQ ID NO: 14 - protein, 67 aa, Artificial Sequence
  source: /note=“Description of Artificial Sequence Synthetic polypeptide”
  MOD_RES (25)..(25): Any amino acid
  MOD_RES (29)..(29): Any amino acid
  MOD_RES (32)..(33): Any amino acid
  MOD_RES (36)..(36): Any amino acid
  MOD_RES (40)..(49): Any amino acid
  SITE (40)..(49): /note=“This region may encompass 1-10 residues”
  MOD_RES (52)..(52): Any amino acid
  MOD_RES (56)..(56): Any amino acid
  VARIANT (58)..(58): /replace=“K” or “R”
  MOD_RES (59)..(60): Any amino acid
  MOD_RES (63)..(63): Any amino acid
  VARIANT (67)..(67): /replace=“K” or “R”
  SITE (1)..(67): /note=“Variant residues given in the sequence have no preference with respect to those in the annotations for variant positions”
  source: /note=“See specification as filed for detailed description of substitutions and preferred embodiments”
VDAKFDKELEEARAKIERLPNLTEXQRRXFIXXLRXDPSXXXXXXXXXXSAXLLAXAAXXNDXQAPA

SEQ ID NO: 15 - protein, 67 aa, Artificial Sequence
  source: /note=“Description of Artificial Sequence Synthetic polypeptide”
  MOD_RES (25)..(25): Any amino acid
  MOD_RES (29)..(29): Any amino acid
  MOD_RES (32)..(33): Any amino acid
  MOD_RES (36)..(36): Any amino acid
  MOD_RES (40)..(49): Any amino acid
  SITE (40)..(49): /note=“This region may encompass 1-10 residues”
  MOD_RES (52)..(52): Any amino acid
  MOD_RES (56)..(56): Any amino acid
  VARIANT (58)..(58): /replace=“K” or “R”
  MOD_RES (59)..(60): Any amino acid
  MOD_RES (63)..(63): Any amino acid
  VARIANT (67)..(67): /replace=“K” or “R”
  SITE (1)..(67): /note=“Variant residues given in the sequence have no preference with respect to those in the annotations for variant positions”
  source: /note=“See specification as filed for detailed description of substitutions and preferred embodiments”
VDAKFDKELEEVRAEIERLPNLTEXQRRXFIXXLRXDPSXXXXXXXXXXSAXLLAXAAXXNDXQAPA

SEQ ID NO: 16 - protein, 67 aa, Artificial Sequence
  source: /note=“Description of Artificial Sequence Synthetic polypeptide”
  MOD_RES (25)..(25): Any amino acid
  MOD_RES (29)..(29): Any amino acid
  MOD_RES (32)..(33): Any amino acid
  MOD_RES (36)..(36): Any amino acid
  MOD_RES (40)..(49): Any amino acid
  SITE (40)..(49): /note=“This region may encompass 1-10 residues”
  MOD_RES (52)..(52): Any amino acid
  MOD_RES (56)..(56): Any amino acid
  VARIANT (58)..(58): /replace=“K” or “R”
  MOD_RES (59)..(60): Any amino acid
  MOD_RES (63)..(63): Any amino acid
  VARIANT (67)..(67): /replace=“K” or “R”
  SITE (1)..(67): /note=“Variant residues given in the sequence have no preference with respect to those in the annotations for variant positions”
  source: /note=“See specification as filed for detailed description of substitutions and preferred embodiments”
VDAKFEKELEEARAEIERLPNLTEXQRRXFIXXLRXDPSXXXXXXXXXXSAXLLAXAAXXNDXQAPA

SEQ ID NO: 17 - protein, 67 aa, Artificial Sequence
  source: /note=“Description of Artificial Sequence Synthetic polypeptide”
  MOD_RES (25)..(25): Any amino acid
  MOD_RES (29)..(29): Any amino acid
  MOD_RES (32)..(33): Any amino acid
  MOD_RES (36)..(36): Any amino acid
  MOD_RES (40)..(49): Any amino acid
  SITE (40)..(49): /note=“This region may encompass 1-10 residues”
  MOD_RES (52)..(52): Any amino acid
  MOD_RES (56)..(56): Any amino acid
  VARIANT (58)..(58): /replace=“K” or “R”
  MOD_RES (59)..(60): Any amino acid
  MOD_RES (63)..(63): Any amino acid
  VARIANT (67)..(67): /replace=“K” or “R”
  SITE (1)..(67): /note=“Variant residues given in the sequence have no preference with respect to those in the annotations for variant positions”
  source: /note=“See specification as filed for detailed description of substitutions and preferred embodiments”
VDAKFDKELEEIRAEIERLPNLTEXQRRXFIXXLRXDPSXXXXXXXXXXSAXLLAXAAXXNDXQAPA

SEQ ID NO: 18 - protein, 67 aa, Artificial Sequence
  source: /note=“Description of Artificial Sequence Synthetic polypeptide”
  MOD_RES (25)..(25): Any amino acid
  MOD_RES (29)..(29): Any amino acid
  MOD_RES (32)..(33): Any amino acid
  MOD_RES (36)..(36): Any amino acid
  MOD_RES (40)..(49): Any amino acid
  SITE (40)..(49): /note=“This region may encompass 1-10 residues”
  MOD_RES (52)..(52): Any amino acid
  MOD_RES (56)..(56): Any amino acid
  VARIANT (58)..(58): /replace=“K” or “R”
  MOD_RES (59)..(60): Any amino acid
  MOD_RES (63)..(63): Any amino acid
  VARIANT (67)..(67): /replace=“K” or “R”
  SITE (1)..(67): /note=“Variant residues given in the sequence have no preference with respect to those in the annotations for variant positions”
  source: /note=“See specification as filed for detailed description of substitutions and preferred embodiments”
VDAKFDKELEEARAEIERLPALTEXQRRXFIXXLRXDPSXXXXXXXXXXSAXLLAXAAXXNDXQAPA

SEQ ID NO: 19 - protein, 5 aa, Artificial Sequence
  source: /note=“Description of Artificial Sequence Synthetic peptide”
GGGGG

SEQ ID NO: 20 - protein, 8 aa, Artificial Sequence
  source: /note=“Description of Artificial Sequence Synthetic peptide”
GGGGGGGG

SEQ ID NO: 21 - protein, 30 aa, Artificial Sequence
  source: /note=“Description of Artificial Sequence Synthetic polypeptide”
  MOD_RES (1)..(1): Any amino acid
  MOD_RES (5)..(5): Any amino acid
  MOD_RES (8)..(9): Any amino acid
  MOD_RES (12)..(12): Any amino acid
  MOD_RES (16)..(16): Any amino acid
  MOD_RES (19)..(19): Any amino acid
  MOD_RES (23)..(23): Any amino acid
  MOD_RES (25)..(27): Any amino acid
  MOD_RES (30)..(30): Any amino acid
XQRRXFIXXLRXDPSXSAXLLAXAXXXNDX

(19) United States
(12) Patent Application Publication
(10) Pub. No.: US 2024/0026344 A1
(43) Pub. Date: Jan. 25, 2024

(54) AFFINITY LIGAND LIBRARIES OF THREE-HELIX BUNDLE PROTEINS AND USES THEREOF

(71) Applicant: Avitide LLC, Lebanon, NH (US)

(72) Inventors: Brandon Kier, Lebanon, NH (US); Molly North, West Lebanon, NH (US); Sarmistha Bhattacharya, Lebanon, NH (US); Thomas Scanlon, Lebanon, NH (US); Warren Kett, West Lebanon, NH (US)

(21) Appl. No.: 18/031,808

(22) PCT Filed: Oct. 13, 2021
(86) PCT No.: PCT/US2021/054874

§ 371 Date: Apr. 13, 2023

Related U.S. Application Data
(60) Provisional application No. 63/188,229, filed on May 13, 2021.
(60) Provisional application No. 63/091,201, filed on Oct. 13, 2020.

(51) Int. Cl. C12N 15/10

(52) U.S. Cl. CPC C12N 15/1037

(57) ABSTRACT

The present disclosure relates to the field of affinity chromatography, and more specifically to the provision of nucleic acid and polypeptide libraries encoding three-helix bundle protein domains suitable for selecting affinity ligands that specifically binds to a target molecule of interest. The disclosure also relates to methods of using those libraries to identify and isolate such affinity ligands to a target molecule.

### CROSS REFERENCE TO RELATED APPLICATIONS

This application claims the benefit of provisional application U.S. Ser. No. 63/091,201, filed on Oct. 13, 2020 and U.S. Ser. No. 63/188,229, filed on May 13, 2021, each of which is incorporated herein by reference in its entirety.

### FIELD OF THE DISCLOSURE

The present disclosure relates to the field of affinity chromatography, and more specifically to the provision of nucleic acid and polypeptide libraries encoding three-helix bundle protein domains suitable for selecting affinity ligands that specifically binds to a target molecule of interest. The disclosure also relates to methods of using those libraries to identify and isolate such affinity ligands to a target molecule.

### BACKGROUND OF THE DISCLOSURE

The purity of biologically-produced therapeutics is tightly scrutinized and regulated by authorities to ensure safety and efficacy. Thus, there remains a need for means to efficiently purify of biologically produced therapeutics to a high degree of purity.

Bioprocess affinity chromatography provides a means to isolate and purify a protein in a few steps, or a single step. However, the development of affinity ligands can be resource intensive and time consuming.

To facilitate discovery of affinity ligands, affinity libraries have been developed that can be rapidly and efficiently screened to identify ligands for a target of interest. Such libraries include libraries based on Protein A domains or the Z domain which are useful to purify immunoglobulins as well as libraries based on antibodies to exploit specific antigen-antibody interactions in purification processes. Further, being able to generate high affinity ligands is another important step in generating affinity agents for bioprocess purification. Ligands should also possess high stability to withstand the rigors of bioprocessing, particularly clean in place (CIP) regimes incorporating NaOH. The ability to reuse resin for many purification cycles has far reaching consequences for the economics of the purification process. The evolution of IgG binding resins based upon protein A exemplify this, with modern variants being able to withstand repeated 0.5 M NaOH CIP cycles. These IgG binding domains are characterized by high binding affinity, typically below 50 nM, to the interface of the CH2 and CH3 domains in the Fc region (Graille et al (2000) Proc Natl Acad Sci USA. 97:5399-5404). Additionally, there also exists a need for affinity ligands devoid of IgG binding, but with the ability to engineer binding towards other modalities that also have high stability. The present disclosure addresses that need. It contemplates a series of polypeptides that can be used as high affinity ligands for modalities or molecules other than IgG.

However, many other biological macromolecules of diverse structures are known with diverse mechanisms underlying protein-ligand interactions and the need remains to find affinity ligands for these macromolecules to take advantage of the streamlining afforded by bioprocess affinity chromatography.

The diversity and mechanisms of molecular interactions is well known (see, e.g., Du et al. (2016) Int. J. Mol. Sci. 17. doi:10.3390/ijms17020144). Examples of some of the structural diversity of protein-ligand interactions that are encountered in biological macromolecules are shown in FIG. 1 and these models highlight the need for and advantage of having a wide array of affinity libraries having different ligand architectures.

Moreover, even within known scaffold libraries, the need for having many different libraries remains. For example, in one study, 48 different scaffold families were interrogated for ligand discovery, and the 62 best ligands were all drawn from a single scaffold library (scaffold 45 from among 48 scaffold libraries). This extraordinary discovery highlights the need for diversity in ligand architectures and not just high sequence diversity within a particular ligand architecture. Efficiently processing ligand diversity between and among scaffold families is the primary technical challenge during ligand discovery efforts. In further work, the sequence similarities among the 62 ligands allowed sorting of those ligands into 5 clades. While other families yielded ˜900 ligands with moderate affinity, those ligands lacked the selectivity associated with the 62 best ligands. Interestingly, the scaffold used for the 62 best ligands was developed to accommodate biological “lock and key” affinity ligands. The dearth of selective ligands from other scaffold types indicated only that this particular scaffold family possessed the molecular architecture required to mimic the cognate receptor (Coyle et al. in Approaches to the Purification, Analysis and Characterization of Antibody-Based Therapeutics, (ed., Matte) Elsevier, 2020, p. 55-79 (at p. 65).

Hence, the scarcity of affinity ligands as well as varying utility among libraries has created a need for ligand libraries capable of interacting with many different types of structures representing multiple ligand-target architectures. The libraries described herein are part of a strategy to address these needs.

### SUMMARY OF THE DISCLOSURE

Affinity ligand libraries are useful as a source of new affinity ligands against target molecules which fill the need for simple and economical ways to purify those targets using bioprocess affinity chromatography.

In one aspect, the disclosure provides a nucleic acid library whose members encode an affinity ligand comprising an amino acid sequence represented by the formula, from N-terminus to C-terminus,

(SEQ ID NO. 1)
[A]-X₁QRRX₂FIX₃X₄LRX₅DPS-[X₆]n-SAX₇LLAX₈AX₉X₁₀X₁₁ND
X₁₂QAPX₁₃-[B],

wherein (a) [A] comprises an α-helix-forming peptide domain;
(b) each of X₁, X₂, X₃, X₄, X₅, X₆, X₇, X₈, X₁₀, X₁₁, and X₁₂ is independently any amino acid;
(c) n represents the number of X₆ residues present and is an integer from one to ten,
(d) each of X₉ and X₁₃ is independently A, K or R; and
(e) [B] is absent, is VD, or is a peptide domain comprising an amino acid sequence of

(SEQ ID NO.9 )
VDGQAGQGGGSGLNDIFEAQKIEWHEHHHHHH,
(SEQ ID NO. 10)
GQAGQGGGSGLNDIFEAQKIEWHEHHHHHH,
(SEQ ID NO. 11)
VDGLNDIFEAQKIEWHEHHHHHH
or
(SEQ ID NO. 12)
GLNDIFEAQKIEWHEHHHHHH

In certain embodiments, the α-helix-forming peptide domain of [A] comprises an alkali-stable helix 1 of a staphylococcal protein A (SPA) domain such as the Z-domain, A-domain, B-domain, C-domain, D-domain or E-domain, and in some embodiments is preferably a Z-domain. In some embodiments, [A] comprises a peptide having an amino acid sequence of VDAKFDKELEEARAEIERLPNLTE (SEQ ID NO. 2), VDAKFDKELEEARAKIERLPNLTE (SEQ ID NO. 3), VDAKFDKELEEVRAEIERLPNLTE (SEQ ID NO. 4), VDAKFEKELEEARAEIERLPNLTE (SEQ ID NO. 5), VDAKFDKELEEIRAEIERLPNLTE (SEQ ID NO. 6) or VDAKFDKELEEARAEIERLPALTE (SEQ ID NO. 7). For any of these embodiments, the N-terminus of [A] can be preceded by M or MAQGT (SEQ ID NO. 8).

In certain embodiments, the nucleic acid library is any one of the libraries of SEQ ID NOS. 13-18 in Table 1. In other embodiments, a nucleic acid library of the disclosure has [A] as VDAKFDKELEEARAEIERLPNLTE (SEQ ID NO. 2), n as one, and X₁₃ as K.

In some embodiments any of the nucleic acid libraries of the disclosure can comprise a peptide tag, optionally, wherein the peptide tag is hemagglutinin, c-myc, a Herpes Simplex virus glycoprotein D, T7, GST, GFP, MBP, a strep-tag, a His-tag, a Myc-tags, a TAP-tag or a FLAG tag.

In certain embodiments, the affinity ligand further comprises a C-terminal lysine or cysteine.

For some embodiments, a nucleic acid library is a phage display library, a yeast display library, an RNA display library or a DNA display library. Phage display libraries are particularly useful and may comprise approximately from 10⁶ to 10⁹ theoretically distinct nucleic acid sequences.

In a further aspect the disclosure provides methods of identifying a polypeptide that interacts selectively with a target molecule of interest which comprises (a) exposing a target molecule of interest to polypeptides produced by expression of a nucleic acid library of the disclosure; and (b) separating polypeptides that selectively interact from those that do not selectively interact with the target molecule. In such a method, the embodiments include having the target molecule of interest expressed on the surface of a phage, bacterium or cell, or attached to, tethered to or otherwise associated with a solid support.

The instant disclosure further provides for methods of screening a library for a polypeptide (i.e., an affinity ligand) that specifically binds with high affinity to a target molecule of interest, the library comprising a plurality of polypeptides produced by expression of a nucleic acid library of the disclosure by (a) incubating a sample of the library with a concentration of a target molecule under conditions suitable for specific binding of the polypeptides to the molecule; (b) incubating a second sample of the library under the same conditions but without target molecule; (c) contacting each of the first and second samples with immobilized target molecule under conditions suitable for binding of the polypeptide to the immobilized target molecule; (d) detecting the polypeptide bound to immobilized target molecule for each sample; and (e) determining the affinity of the polypeptide for the target molecule by calculating the ratio of the amounts of bound polypeptide from the first sample over the amount bound polypeptide from the second sample.

A still further aspect of the disclosure relates to methods of identifying one or more affinity ligands that specifically bind with a target molecule of interest which comprises: (a) contacting the target molecule with a phage display library; (b) separating phage that specifically bind with (or to) the target molecule from those that do not selectively bind with the target molecule to produce an enriched phage library; (c) repeating steps a) and b) using the enriched phage library to produce a further enriched phage library; (d) repeating step c) until the further enriched phage library is enriched from at least about 10- to about 10⁶-fold or more relative to the original phage library; and (e) plating the further enriched phage library and isolating and characterizing individual clones therefrom and to thereby identify one or more affinity ligands that specifically bind to the target molecule of interest.

In some embodiments of the foregoing method, the target molecule is bound to or attached to a solid support. In other embodiments, the phage display library is bound to or attached to a solid support. In either case, the target molecule can be an adeno-associated virus (AAV) or AAV capsid, and more particularly, the AAV is AAV8 or an AAV8 serotype variant.

In a further aspect, the present disclosure provides polypeptide library compositions comprising a plurality of synthetic or recombinant polypeptides, each polypeptide comprising an affinity ligand of the nucleic acid libraries described herein.

In another aspect, the disclosure relates to methods of identifying a polypeptide that binds specifically to a target molecule of interest which comprises: (a) exposing a target molecule of interest to a polypeptide library composition of the disclosure; (b) separating polypeptides that specifically bind to the target molecule from those that do not selectively bind the target molecule; and (c) identifying one or more of the polypeptides bound by the target molecule.

### BRIEF DESCRIPTION OF THE DRAWINGS

FIG. 1A to FIG. 1F depict space filling and ribbon models of different target-ligand interactions, including (A) a large groove interface, (B) a large planar interface, (C) surface-surface complementarity, (D) shallow pocket binding, (E) convex/concave (protruding loops) binding, and (F) deep pocket binding.

FIG. 2A and FIG. 2B show ribbon representations of the addition of a helical forming sequence to the N-terminus of a sequence that has the ability to form 2-helical domains (A). This addition generates a 3-helix bundle protein (B). In the representation shown in (A), what would be helices 3 and 2 of a 3-helix bundle protein are shown left to right, making the N-terminus of helix 2 appear on the right side In the representation shown in in (B), the helices appear in order as helix 3, helix 1 and helix 2, with the N-terminus of helix 1 appear at the center top of the diagram.

FIG. 3 shows a ribbon representation of the addition of a long loop (highlighted as darker section) between helix 3 and helix 2 of FIG. 2B.

FIG. 4 shows a sensorgram for an exemplary affinity agent.

FIG. 5 shows exemplary stability data for certain affinity agents in the presence of 0.5 M NaOH.

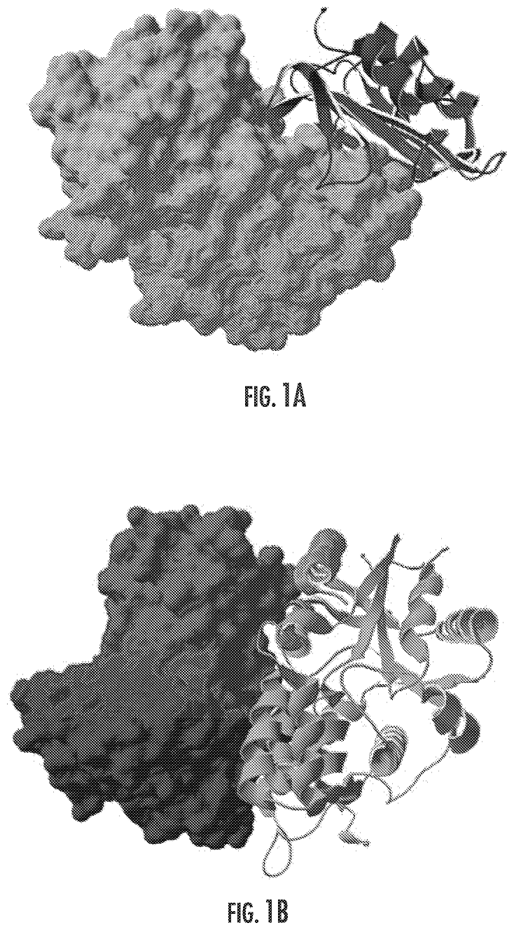

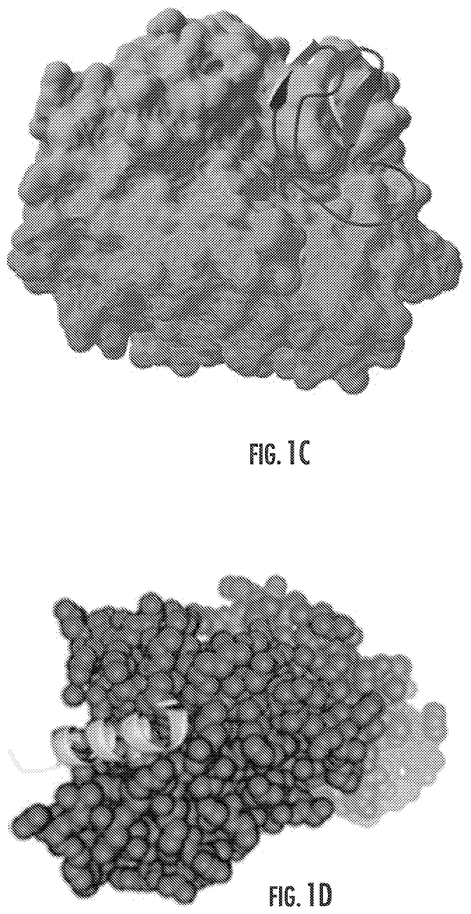

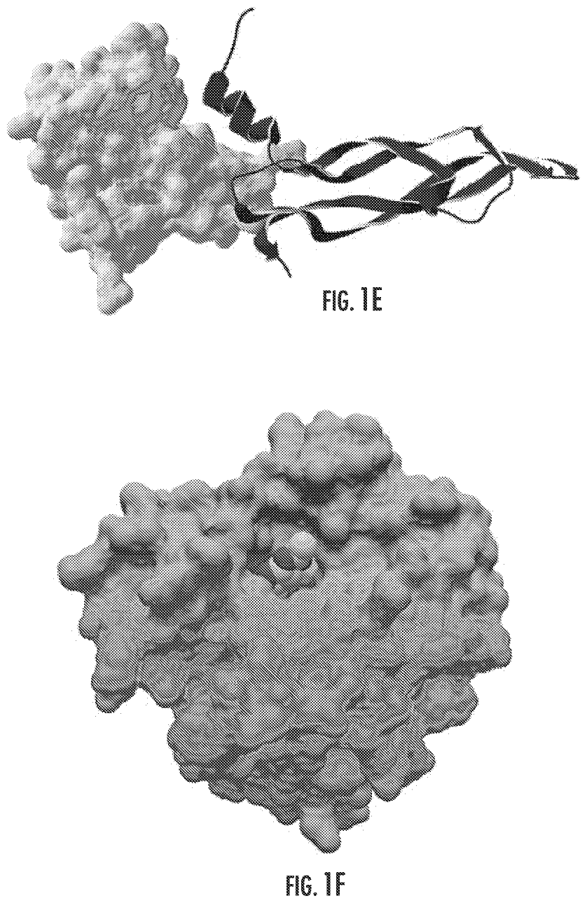

### DETAILED DESCRIPTION OF THE DISCLOSURE

### Definitions

In order for the present disclosure to be more readily understood, certain terms are defined below. Unless defined otherwise herein, technical and scientific terms used herein have the same meaning as commonly understood by one of ordinary skill in the art to which this disclosure is related.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or embodiments of the disclosure, which can be had by reference to the specification as a whole. Accordingly, the terms defined immediately below are more fully defined by reference to the specification in its entirety.

It is to be noted that the term “a” or “an” entity refers to one or more of that entity; for example, “an affinity ligand” is understood to represent one or more affinity ligands. As such, the terms “a” (or “an”), “one or more,” and “at least one” can be used interchangeably herein.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The term “plurality” as used herein refers to the number of members of a collection, which minimum is at least 10, 20, 30, 50, 75, 100, 1000 or more, and which minimum or maximum number may not be readily ascertainable, but which may be indicated by type of collection or the context of its use. For example, a phage display library contains a plurality of phage equal to its titer (which may be the same or different), and by extension encodes a corresponding plurality of polypeptides.

The term “including” is used to mean “including but not limited to.” “Including” and “including but not limited to” are used interchangeably.

Biologically active: As used herein, the term “biologically active” refers to a characteristic of any agent that has activity in a biological system, and particularly in an organism. For instance, an agent that, when administered to an organism, has a biological or physiological effect on that organism, is considered to be biologically active.

Variant and Mutant: The term “variant” is usually defined in the scientific literature and used herein in reference to an organism that differs genetically in some way from an accepted standard, “Variant” can also be used to describe phenotypic differences that are not genetic (King and Stansfield, 2002, A dictionary of genetics, 6th ed., New York, New York, Oxford University Press.

The term “mutation” is defined by most dictionaries and used herein in reference to the process that introduces a heritable change into the structure of a gene (King & Stansfield, 2002) thereby producing a “mutant.” The term “variant” is increasingly being used in place of the term “mutation” in the scientific and non-scientific literature. The terms are used interchangeably herein.

Conservative and non-conservative substitution: A “conservative” amino acid substitution is one in which one amino acid residue is replaced with another amino acid residue having a similar side chain. Families of amino acid residues having similar side chains have been defined in the art, including basic side chains (e.g., lysine (K), arginine (R), histidine (H)); acidic side chains (e.g., aspartic acid (D), glutamic acid (E)); uncharged polar side chains (e.g., glycine (G); asparagine (N), glutamine (Q), serine (S), threonine (T), tyrosine (Y), cysteine (C)); nonpolar side chains (e.g., alanine (A), valine (V), leucine (L), isoleucine (I), proline (P), phenylalanine (F), methionine (M), tryptophan (W), beta-branched side chains (e.g., threonine (T), valine (V), isoleucine (I)); and aromatic side chains (e.g., tyrosine (Y), phenylalanine (F), tryptophan (W), histidine (H)). For example, substitution of a phenylalanine for a tyrosine is a conservative substitution. In some embodiments, conservative amino acid substitutions in the sequence of a ligand confer or improve specific binding of the ligand a target of interest. In some embodiments, conservative amino acid substitutions in the sequences of a ligand do not reduce or abrogate the binding of the ligand to a target of interest. In some embodiments, conservative amino acid substitutions do not significantly affect specific binding of a ligand to a target of interest. Methods of identifying nucleotide and amino acid conservative substitutions and non-conservative substitutions which confer, alter or maintain selective binding affinity are known in the art (see, e.g., Brummell, Biochem. 32:1180-1187 (1993); Kobayashi, Protein Eng. 12(10):879-884 (1999); and Burks, PNAS 94:412-417 (1997)). In some embodiments, non-conservative amino acid substitutions in the sequence of a ligand confer or improve specific binding of the ligand a target of interest. In some embodiments, non-conservative amino acid substitutions in the sequences of a ligand do not reduce or abrogate the binding of the ligand to a target of interest. In some embodiments, non-conservative amino acid substitutions do not significantly affect specific binding of a ligand to a target of interest.

Affinity chromatography: As used herein the term “affinity chromatography” refers to the specific mode of chromatography in which an affinity ligand interacts with a target via biological affinity in a “lock-key” fashion. Examples of useful interactions in affinity chromatography are e.g., enzyme-substrate interaction, biotin-avidin interaction, antibody-antigen interaction, etc.

Affinity ligand and Ligand: The terms “affinity ligand” and “ligand” are used interchangeably herein. These terms are used herein to refer to molecules that are capable of reversibly binding with high affinity to a moiety specific for it, e.g., a polypeptide or protein.

Protein-based ligand: The term “protein-based ligands” as used herein means ligands which comprise a peptide or protein or a part of a peptide or protein that binds reversibly to a target polypeptide or protein. It is understood that the “ligands” of the disclosure are protein-based ligands.

Affinity agent: As used herein, the term “affinity agent” is in reference to a solid support or matrix to which a biospecific affinity ligand is covalently attached. Typically, the solid support or matrix is insoluble in the system in which the target molecule is purified. The terms “affinity agent” and “affinity separation matrix(ces)” and “separation matrix(ces)” are used interchangeably herein.

Linker: As used herein a “linker” refers to a peptide or other chemical linkage that functions to link otherwise independent functional domains. In some embodiments, a linker is located between a ligand and another polypeptide component containing an otherwise independent functional or structural domain. In some embodiments, a linker is a peptide or other chemical linkage located between a ligand and a surface.

Naturally occurring: The term “naturally occurring” when used in connection with biological materials such as a nucleic acid molecules, polypeptides, and host cells, refers to those which are found in nature and not modified by a human being. Conversely, “non-natural” or “synthetic” when used in connection with biological materials refers to those which are not found in nature and/or have been modified by a human being.

“Non-natural amino acids,” “amino acid analogs” and “non-standard amino acid residues” are used interchangeably herein. Non-natural amino acids that can be substituted in a ligand as provided herein are known in the art. In some embodiments, a non-natural amino acid is 4-hydroxyproline which can be substituted for proline; 5-hydroxylysine which can be substituted for lysine; 3-methylhistidine which can be substituted for histidine; homoserine which can be substituted for serine; and ornithine which can be substituted for lysine. Additional examples of non-natural amino acids that can be substituted in a polypeptide ligand include, but are not limited to molecules such as: D-isomers of the common amino acids, 2,4-diaminobutyric acid, alpha-amino isobutyric acid, A-aminobutyric acid, Abu, 2-amino butyric acid, gamma-Abu, epsilon-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, beta-alanine, lanthionine, dehydroalanine, γ-aminobutyric acid, selenocysteine and pyrrolysine fluoro-amino acids, designer amino acids such as beta-methyl amino acids, C alpha-methyl amino acids, and N alpha-methyl amino acids.

“Polynucleotide” and “nucleic acid molecule”: As used interchangeably herein, polynucleotide and nucleic acid molecule refer to a polymeric form of nucleotides of any length, either ribonucleotides or deoxyribonucleotides. These terms include, but are not limited to, DNA, RNA, cDNA (complementary DNA), mRNA (messenger RNA), rRNA (ribosomal RNA), shRNA (small hairpin RNA), snRNA (small nuclear RNA), snoRNA (short nucleolar RNA), miRNA (microRNA), genomic DNA, synthetic DNA, synthetic RNA, and/or tRNA (transfer RNA).

Operably linked: The term “operably linked,” as used herein, indicates that two or more components are arranged such that the components function normally and allow the possibility that at least one of the components can mediate a function that is exerted upon at least one of the other components. Two molecules are “operably linked” whether they are attached directly or indirectly.

Peptide tag: The term “peptide tag” as used herein refers to a peptide sequence that is part of or attached (for instance through genetic engineering) to another protein, to provide a function to the resultant fusion. Peptide tags are usually relatively short in comparison to a protein to which they are fused. In some embodiments, a peptide tag is four or more amino acids in length, such as, 6, 7, 8, 9, 10, 15, 20, or 25 or more amino acids. In some embodiments, a ligand is a protein that contains a peptide tag. Numerous peptide tags that have uses as provided herein are known in the art. Examples of peptide tags that may be a component of a ligand fusion protein or a target bound by a ligand (e.g., a ligand fusion protein) include but are not limited to HA (hemagglutinin), c-myc, the Herpes Simplex virus glycoprotein D (gD), T7, GST, GFP, MBP, Strep-tags, His-tags, Myc-tags, TAP-tags and FLAG tag (Eastman Kodak, Rochester, N.Y.) Likewise, antibodies to the tag epitope allow detection and localization of the fusion protein in, for example, affinity purification, Western blots, ELISA assays, and immunostaining of cells.

Polypeptide: The term “polypeptide” as used herein refers to a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Specifically binds: As used herein in reference to ligands, the term “specifically binds” or “has selective affinity for” means a ligand reacts or associates more frequently, more rapidly, with greater duration, with greater affinity, or combinations of the above to a particular epitope, protein, or target molecule than with alternative substances, including unrelated proteins. Because of the sequence identity between homologous proteins in different species, specific binding can include a binding agent that recognizes a protein or target in more than one species, e.g., is bi- or tri-specific. Likewise, because of homology within certain regions of polypeptide sequences of different proteins, specific binding can include a binding agent that recognizes more than one protein or target. It is understood that, in certain embodiments, a binding agent that specifically binds a first target may or may not specifically bind a second target. As such, “specific binding” does not necessarily require (although it can include) exclusive binding, i.e., binding to a single target. Thus, a ligand or affinity agent may, in certain embodiments, specifically bind more than one target. In certain embodiments, multiple targets may be bound by the same binding site on an affinity agent. “Selectively binds” or “selectively interacts” is used herein interchangeably with “specifically binds.”

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

The present disclosure encompasses, inter alia, nucleic acid and polypeptide libraries for selection of an affinity ligand specific for one or more targets of interest, for example, in some embodiments, virus particles.

### Affinity Ligand Libraries, Construction Thereof and Methods of Use

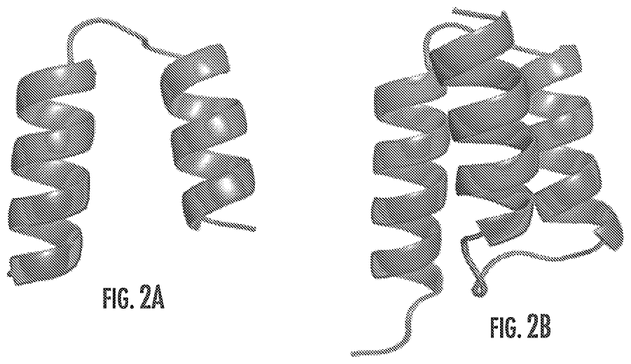

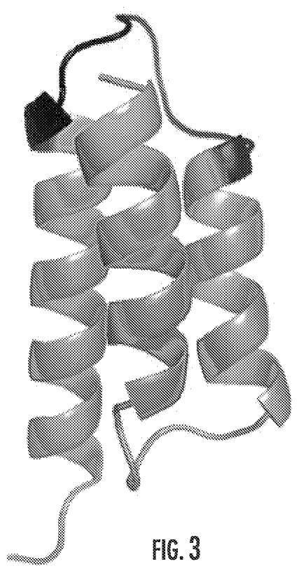

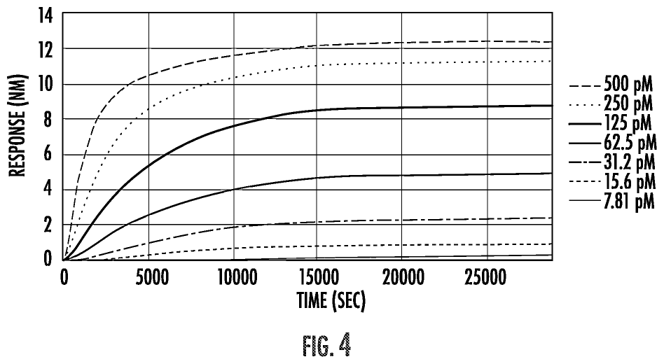

Libraries of the disclosure comprise polypeptide compositions and/or nucleic acid molecules encoding certain three-bundle helical proteins (FIGS. 2 and 3) and can be used to identify and select sequences within those libraries which bind selectively to one or more target molecules of interest to thereby yield affinity ligands for use in bioprocessing of a target molecule or for other uses. Libraries of the disclosure enable the generation of novel affinity ligands, which in some embodiments are alkali stable and bind selectively with high affinity to a select target of interest.

Methods to make the libraries disclosed herein include but are not limited to, direct synthesis, recombinant production, dimer-trimer or codon mutagenesis, site-directed mutagenesis and the like and any combinations thereof for nucleic acid libraries, and for peptide libraries, direct chemical synthesis. All such methods are well known in the art.

Accordingly, the nucleic acid libraries of the disclosure comprise members which encode an affinity ligand comprising an amino acid sequence represented by the formula, from N-terminus to C-terminus,

(SEQ ID NO. 1)
[A]-X₁QRRX₂FIX₃X₄LRX₅DPS-[X₆]n-SAX₇LLAX₈AX₉X₁₀X₁₁N
DX₁₂QAPX₁₃-[B],

wherein (a) [A] comprises an α-helix-forming peptide domain;
(b) each of X₁, X₂, X₃, X₄, X₅, X₆, X₇, X₈, X₁₀, X₁₁, and X₁₂ is independently any amino acid;
(c) n represents the number of X₆ residues present and is an integer from one to ten,
(d) each of X₉ and X₁₃ is independently A, K or R; and
(e) [B] is absent, is VD, or is a peptide domain comprising an amino acid sequence of

(SEQ ID NO.9 )
VDGQAGQGGGSGLNDIFEAQKIEWHEHHHHHH,
(SEQ ID NO. 10)
GQAGQGGGSGLNDIFEAQKIEWHEHHHHHH,
(SEQ ID NO. 11)
VDGLNDIFEAQKIEWHEHHHHHH
or
(SEQ ID NO. 12)
GLNDIFEAQKIEWHEHHHHHH,

Moiety [A] of the formula comprises an α-helix-forming peptide domain and is preferably alkali stable. Such peptide domains are well known in the art from a variety of sources. In some embodiments, the α-helix-forming peptide domains staphylococcus Protein A (SPA) domains. In certain embodiments, the SPA domain comprises an alkali-stable helix 1 of the SPA domain found at residues 5-19 of any one of an SPA Z-domain, A-domain, B-domain, C-domain, D-domain or E-domain, and preferably is that of a Z-domain. (see, e.g., Nilsson et al. (1987) Prot. Eng. 1:107-113), and U.S. Pat. Nos. 6,534,628, 6,831,161, 7,834,158, 9,187,555, 9,663,558, 9,683,013, 10,308,690, 10,501,557, and 10,703,774).

In some embodiments, the libraries of the disclosure, [A] comprises a peptide having an amino acid sequence of VDAKFDKELEEARAEIERLPNLTE (SEQ ID NO. 2), VDAKFDKELEEARAKIERLPNLTE (SEQ ID NO. 3), VDAKFDKELEEVRAEIERLPNLTE (SEQ ID NO. 4), VDAKFEKELEEARAEIERLPNLTE (SEQ ID NO. 5), VDAKFDKELEEIRAEIERLPNLTE (SEQ ID NO. 6) or VDAKFDKELEEARAEIERLPALTE (SEQ ID NO. 7). The N-terminus of the affinity ligands in the library (i.e., the N-terminus of [A]) may be a methionine or may include an additional amino acid sequence of MAQGT (SEQ ID NO. 8).

In embodiments of the nucleic acid libraries of the disclosure wherein [B] is absent, or is VD, additional amino acids can be present at the C terminus of [B]. Such additional amino acids include peptide tags and amino acids to facilitate coupling of the affinity ligand to a support matrix.

Hence, any of the nucleic acid libraries of the disclosure can further comprise a peptide tag, including but not limited to, a peptide tag which is hemagglutinin, c-myc, a Herpes Simplex virus glycoprotein D, T7, GST, GFP, MBP, a Strep-tag, a His-tag, a Myc-tags, a TAP-tag or a FLAG tag.

In certain embodiments, the nucleic acid libraries are any one of the libraries provided in Table 1, i.e., the library comprise members which encode an affinity ligand comprising an amino acid sequence set forth in Table 1, wherein the X's, [A], n and [B] are as defined above. In some embodiments of the libraries, n is 1. Because X₆ may form part of a loop structure, it is preferred that X₆ is not P or H. In an embodiment, the nucleic acid libraries of the disclosure have the formula above wherein [A] is VDAKFDKELEEARAEIERLPNLTE (SEQ ID NO. 2), n is one, and X₁₃ is K.

TABLE 1

| SEQ | |
|---|---|
| ID NO. | LIBRARY SEQUENCE |
| 13 | VDAKFDKELEEARAEIERLPNLTEX₁QRRX₂FIX₃X₄LRX₅ |
| | DPS-[X₆]ₙ-SAX₇LLAX₈AX₉X₁₀X₁₁NDX₁₂QAPX₁₃ |
| 14 | VDAKFDKELEEARAKIERLPNLTEX₁QRRX₂FIX₃X₄LRX₅ |
| | DPS-[X₆]ₙ-SAX₇LLAX₈AX₉X₁₀X₁₁NDX₁₂QAPX₁₃ |
| 15 | VDAKFDKELEEVRAEIERLPNLTEX₁QRRX₂FIX₃X₄LRX₅ |
| | DPS-[X₆]ₙ-SAX₇LLAX₈AX₉X₁₀X₁₁NDX₁₂QAPX₁₃ |
| 16 | VDAKFEKELEEARAEIERLPNLTEX₁QRRX₂FIX₃X₄LRX₅ |
| | DPS-[X₆]ₙ-SAX₇LLAX₈AX₉X₁₀X₁₁NDX₁₂QAPX₁₃ |
| 17 | VDAKEDKELEEIRAEIERLPNLTEX₁QRRX₂FIX₃X₄LRX₅ |
| | DPS-[X₆]ₙ-SAX₇LLAX₈AX₉X₁₀X₁₁NDX₁₂QAPX₁₃ |
| 18 | VDAKFDKELEEARAEIERLPALTEX₁QRRX₂FIX3X₄LRX₅ |
| | DPS-[X₆]ₙ-SAX₇LLAX₈AX₉X₁₀X₁₁NDX₁₂QAPX₁₃ |

In an exemplary embodiment for the libraries, [A] is VDAKFDKELEEARAEIERLPNLTE; each of the X positions other than X₉ is any amino acid; and X₉ is A, R or K; provided that at least two of the following criteria are met: X₂ is not alanine (A), X₄ is not serine (S), X₅ is not aspartic acid (D), X₆ is not glutamine or serine (Q or S), X₈ is not glutamic acid (E), X₁₀ is not lysine (L) and X₁₁ is not leucine (L).

In another exemplary embodiment for the libraries, [A] is VDAKFDKELEEARAEIERLPNLTE, each of the X positions other than X₉ and X₁₂ is any amino acid, and each of X₉ and X₁₂ are A, R or K; provided that at least two of the following criteria are met: X₂ is not alanine (A), X₄ is not serine (S), X₅ is not aspartic acid (D), X₆ is not glutamine or serine (Q or S), X₈ is not glutamic acid (E), X₁₀ is not lysine (L) and X₁₁ is not leucine (L).

In some embodiments, any of the nucleic acid libraries of the disclosure can further comprise a peptide tag, including but not limited to, wherein the peptide tag is hemagglutinin, c-myc, a Herpes Simplex virus glycoprotein D, T7, GST, GFP, MBP, a strep-tag, a His-tag, a Myc-tags, a TAP-tag or a FLAG tag. In other embodiments, either in conjunction with a peptide tag or not, the affinity ligand in the library comprises a C-terminal lysine or cysteine.

In some embodiments, the nucleic acid library of the disclosure is a phage display library, a yeast display library, an RNA display library or a DNA display library. In phage display libraries, it is possible to have from 10⁶ to 10⁹ theoretically distinct nucleic acid sequences.

In accordance with another aspect of the disclosure, the nucleic acid libraries hereof are used in various methods for identifying polypeptides that bind selectively to target molecules of interest. An embodiment provides a method of identifying a polypeptide that binds selectively to a target molecule of interest which comprises: (a) exposing a target molecule of interest to polypeptides produced by expression of a nucleic acid library of the disclosure; and (b) separating polypeptides that selectively bind from those that do not selectively bind the target molecule. In an embodiment, the target molecule of interest is expressed on the surface of a phage, bacterium or cell, or is attached to, tethered to or otherwise associated with a solid support.

Another embodiment provides a method of screening a library for a polypeptide that selectively binds with high affinity to a target molecule of interest, the library comprising a plurality of polypeptides produced by expression of a nucleic acid library of the disclosure and comprises: (a) incubating a sample of the library with a concentration of a target molecule under conditions suitable for specific binding of the polypeptides to the molecule; (b) incubating a second sample of the library under the same conditions but without target molecule; (c) contacting each of the first and second samples with immobilized target molecule under conditions suitable for binding of the polypeptide to the immobilized target molecule; (d) detecting the polypeptide bound to immobilized target molecule for each sample; (e) determining the affinity of the polypeptide for the target molecule by calculating the ratio of the amounts of bound polypeptide from the first sample over the amount bound polypeptide from the second sample.

Yet another embodiment provides a method of identifying one or more affinity ligands that selectively bind to a target molecule of interest which comprises: (a) contacting said target molecule with a phage display library of the disclosure; (b) separating phage that selectively bind the target molecule from those that do not selectively bind the target molecule to produce an enriched phage library; (c) repeating steps (a) and (b) with the enriched phage library to produce a further enriched phage library; (d) repeating step (c) until the further enriched phage library is enriched from at least about 10- to about 10⁶-fold or more relative to the original phage library; and (e) plating the further enriched phage library and isolating and characterizing individual clones therefrom to thereby identify one or more affinity ligands that selectively bind to the target molecule of interest. The number of cycles needed to obtain a sufficiently further enriched phage library to readily isolate the desired, individual clones typically ranges from three to eight rounds of selection and more typically can be done with 3-4 rounds of selection. In this method, either the target molecule or the phage display library can be bound to or attached to a solid support to facilitate selective binding (and simplify wash conditions, which stringency can be varied in successive rounds (see, the Examples). Any method known in the art for eluting and recovering bound phage can be used.

In preferred embodiments for any of these methods, the target molecule is an AAV virus or capsid, and preferably an AAV8 virus or serotype variant thereof.

A still further aspect relates to a polypeptide library composition comprising a plurality of synthetic or recombinant polypeptides, each polypeptide comprising an affinity ligand as defined above for any on the nucleic acid libraries of the disclosure. In some embodiments, a plurality is 50 or more or as defined herein (see above). In those embodiments of the polypeptide compositions produced by a phage display library, the composition can have from 100 to 10¹⁰ polypeptides as determined by the phage titer.

Yet a further aspect relates to a method of identifying a polypeptide (affinity ligand) that binds selectively to a target molecule of interest which comprises (a) exposing a target molecule of interest to a polypeptide library composition of the disclosure; and (b) separating polypeptides that selectively bind to said target molecule from those that do not selectively bind the target molecule. Accordingly, the disclosure provides powerful methods for screening and selecting a affinity ligand with binding specificity directed to one or more of any number of desired molecular target molecules. The libraries of the disclosure may be screened and clones comprising putative binding moiety sequences (polypeptide and/or nucleic acid) may be enriched, purified and tested in any in vitro or in vivo biological assays known and available to the art for the particular molecular target molecule of interest. Once molecular target-binding clones are isolated, polypeptide and/or nucleic acid molecules encoding the affinity ligand may be identified and optionally isolated. One of skill in the art can use standard genetic and molecular engineering, e.g., affinity maturation and other well-known techniques to optimize the characteristics of the binding moiety for its intended purpose, e.g., to produce an affinity ligand that interacts with and reversibly binds to a target of interest with high affinity to facilitate purification of that target and bioprocess manufacturing. In general, the affinity ligands of the disclosure are alkali stable.

### Ligand Binding to Targets of Interest for Use in an Affinity Agent

The characteristics of a ligand binding to a target can be determined using known or modified assays, bioassays, and/or animal models known in the art for evaluating such activity.

As used herein, terms such as “binding affinity for a target”, “binding to a target” and the like refer to a property of a ligand which may be directly measured, for example, through the determination of affinity constants (e.g., the amount of ligand that associates and dissociates at a given antigen concentration). Several methods are available to characterize such molecular interactions, for example, competition analysis, equilibrium analysis and microcalorimetric analysis, and real-time interaction analysis based on surface plasmon resonance interaction (for example using a BIACORE instrument). These methods are well-known to those of skill in the art and are discussed in publications such as Neri D et al. (1996) Tibtech 14:465-470 and Jansson M et al. (1997) J Biol Chem 272:8189-8197.

Affinity requirements for a given ligand binding event are contingent on a variety of factors including, but not limited to, the composition and complexity of the binding matrix, the valency and density of both the ligand and target molecules, and the functional application of the ligand. In some embodiments, a ligand binds a target of interest with a dissociation constant (K_{D}) of less than or equal to 5×10⁻³ M, 10⁻³ M, 5×10⁻⁴ M, 10⁻⁴ M, 5×10⁻⁵ M, or 10⁻⁵ M. In some embodiments, a ligand binds a target of interest with a K_{D} of less than or equal to 5×10⁻⁶ M, 10⁻⁶ M, 5×10⁻⁷ M, 10⁻⁷ M, 5×10⁻⁸ M, or 10⁻⁸ M. In some embodiments, a ligand binds a target of interest with a K_{D} less than or equal to 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M. In some embodiments, a ligand generated by methods disclosed herein has a dissociation constant of from about 10⁻⁴ M to about 10⁻⁵ M, from about 10⁻⁵ M to about 10⁻⁶ M, from about 10⁻⁶ M to about 10⁻⁷ M, from about 10⁻⁷ M to about 10⁻⁸ M, from about 10⁻⁸ M to about 10⁻⁹ M, from about 10⁻⁹ M to about 10⁻¹⁰ M, from about 10⁻¹⁰ M to about 10⁻¹¹ M, or from about 10⁻¹¹ M to about 10⁻¹² M.

Binding experiments to determine K_{D} and off-rates can be performed in a number of conditions. The buffers in which to make these solutions can readily be determined by one of skill in the art and depend largely on the desired pH of the final solution. Low pH solutions (<pH 5.5) can be made, for example, in citrate buffer, glycine-HCl buffer, or in succinic acid buffer. High pH solutions can be made, for example, in Tris-HCl, phosphate buffers, or sodium bicarbonate buffers. A number of conditions may be used to determine K_{D} and off-rates for the purpose of determining, for example, optimal pH and/or salt concentrations.

In some embodiments, a ligand specifically binds a target of interest with a k_{off} ranging from 0.1 to 10⁻⁷ sec⁻¹, 10⁻² to 10⁻⁷ sec⁻¹, or 0.5×10⁻² to 10⁻⁷ sec⁻¹. In some embodiments, a ligand binds a target of interest with an off rate (k_{off}) of less than 5×10⁻² sec⁻¹, 10⁻² sec⁻¹, 5×10⁻³ sec⁻¹, or 10⁻³ sec⁻¹. In some embodiments a ligand binds a target of interest with an off rate (k_{off}) of less than 5×10⁻⁴ sec⁻¹, 10⁻⁴ sec⁻¹, 5×10⁻⁵ sec⁻¹, or 10⁻⁵ sec⁻¹, 5×10⁻⁶ sec⁻¹, 10⁻⁶ sec⁻¹, 5×10⁻⁷ sec⁻¹, or 10⁻⁷ sec⁻¹.

In some embodiments, a ligand specifically binds a target of interest with a kₒₙ ranging from about 10³ to 10⁷ M⁻¹ sec⁻¹, 10³ to 10⁶ M⁻¹ sec⁻¹, or 10³ to 10⁵ M⁻¹sec⁻¹. In some embodiments, a ligand (e.g., a ligand fusion protein) binds the target of interest with an on rate (kₒₙ) of greater than 10³ M⁻¹sec⁻¹, 5×10³ M⁻¹sec⁻¹, 10⁴ M⁻¹sec⁻¹, or 5×10⁴ M⁻¹sec⁻¹. In an additional embodiment, a ligand, binds a target of interest with a kₒₙ of greater than 10⁵ M⁻¹sec⁻¹, 5×10⁵ M⁻¹sec⁻¹, 10⁶ M⁻¹sec⁻¹, 5×10⁶ M⁻¹ sec⁻¹, or 10⁷ M⁻¹ sec⁻¹.

### Targets of Interest

In accordance with various embodiments, a target of interest specifically bound by a ligand can be any molecule for which it is desirable for a ligand of an affinity agent to bind. For example, a target specifically bound by ligand can be any target of purification, manufacturing, formulation, therapeutic, diagnostic, or prognostic relevance or value. Non-limiting uses include therapeutic and diagnostic uses. A number of exemplary targets are provided herein, by way of example, and are intended to be illustrative and not limiting. A target of interest can be naturally occurring or synthetic. In some embodiments, a target is a biologically active protein. In some embodiments, a target of interest is an extracellular component or an intracellular component, a soluble factor (e.g., an enzyme, hormone, cytokine, growth factor, antibody, and the like), or a transmembrane protein (e.g., a cell surface receptor). In some embodiments, a target of interest specifically bound by a ligand is itself a ligand having a different sequence.

### Linkers

The terms “linker” and “spacer” are used interchangeably herein to refer to a peptide or other chemical linkage that functions to link otherwise independent functional domains. In some embodiments, a linker is located between a ligand and another polypeptide component containing an otherwise independent functional domain. Suitable linkers for coupling two or more linked ligands may generally be any linker used in the art to link peptides, proteins or other organic molecules. In some embodiments, such a linker is suitable for constructing proteins or polypeptides that are intended for pharmaceutical use.

Suitable linkers for operably linking a ligand and an additional component of a ligand fusion protein in a single-chain amino acid sequence include but are not limited to, polypeptide linkers such as glycine linkers, serine linkers, mixed glycine/serine linkers, glycine- and serine-rich linkers or linkers composed of largely polar polypeptide fragments.

In some embodiments, a linker comprises a majority of amino acids selected from glycine, alanine, proline, asparagine, glutamine, and lysine. In some embodiments, a linker comprises a majority of amino acids selected from glycine, alanine, proline, asparagine, aspartic acid, threonine, glutamine, and lysine. In some embodiments, a ligand linker is made up of a majority of amino acids that are sterically unhindered. In some embodiments, a linker comprises a majority of amino acids selected from glycine, serine, and/or alanine. In some embodiments, a peptide linker is selected from polyglycines (such as (Gly)₅, and (Gly)₈, poly(Gly-Ala), and polyalanines.

Linkers can be of any size or composition so long as they are able to operably link a ligand in a manner that permits the ligand to bind a target of interest. In some embodiments, linkers are from about 1 to 50 amino acids, from about 1 to 20 amino acids, from about 1 to 15 amino acids, from about 1 to 10 amino acids, from about 1 to 5 amino acids, from about 2 to 20 amino acids, from about 2 to 15 amino acids, from about 2 to 10 amino acids, or from about 2 to 5 amino acids. It should be clear that the length, the degree of flexibility and/or other properties of the linker(s) may influence certain properties of a ligand for use in an affinity agent, such as affinity, specificity or avidity for a target of interest, or for one or more other target proteins of interest, or for proteins not of interest (i.e., non-target proteins). In some embodiments, two or more linkers are utilized. In some embodiments, two or more linkers are the same. In some embodiments, two or more linkers are different.

In some embodiments, a linker is a non-peptide linker such as an alkyl linker, or a PEG linker. For example, alkyl linkers such as —NH—(CH2)s-C(0)-, wherein s=2-20 can be used. These alkyl linkers may further be substituted by any non-sterically hindering group such as lower alkyl e.g., C1 C6) lower acyl, halogen (e.g., CI, Br), CN, NH2, phenyl, etc. An exemplary non-peptide linker is a PEG linker. In some embodiments, a PEG linker has a molecular weight of from about 100 to 5000 kDa, or from about 100 to 500 kDa.

Linkers can be evaluated using techniques described herein and/or otherwise known in the art. In some embodiments, linkers do not alter (e.g., do not disrupt) the ability of a ligand to bind a target molecule.

### Affinity Agents Comprising Conjugated Ligands: Affinity Separation Matrices

Ligands or that promote specific binding to targets of interest can be chemically conjugated to a variety of surfaces used in chromatography, e.g., beads, resins, gels, membrane, monoliths, etc., to prepare an affinity agent. Affinity agents comprising ligands against targets of interest are useful for purification and manufacturing applications.

In some embodiments, a ligand (e.g., a ligand fusion protein) contains at least one reactive residue. Reactive residues are useful, for example, as sites for the attachment of conjugates such as chemotherapeutic drugs or diagnostic agents. Exemplary reactive amino acid residues include lysine and cysteine, for example. A reactive residue can be added to a ligand at either end, or within the ligand sequence and/or can be substituted for another amino acid in the sequence of a ligand. A suitable reactive residue (e.g., lysine, cysteine, etc.) can also be located within the sequence of an identified ligand without need for addition or substitution.

### Attachment to a Solid Surface

“Solid surface,” “support,” or “matrix” are used interchangeably herein and refer to, without limitation, any column (or column material), bead, test tube, microtiter dish, solid particle (for example, agarose or Sepharose), microchip (for example, silicon, silicon-glass, or gold chip), or membrane (synthetic (e.g. a filter) or biological (e.g. liposome or vesicle)) to which a ligand or other protein may be attached (i.e., coupled, linked, or adhered), either directly or indirectly (for example, through other binding partner intermediates such as a linker), or in which a ligand may be embedded (for example, through a receptor or channel). Reagents and techniques for attaching polypeptides to solid supports are well known in the art, e.g., carbamate or thiolether coupling. Suitable solid supports include, but are not limited to, a chromatographic resin or matrix (e.g., agarose beads such as Sepharose-4 FF agarose beads), the wall or floor of a well in a plastic microtiter dish, a silica-based biochip, polyacrylamide, agarose, silica, nitrocellulose, paper, plastic, nylon, metal, and combinations thereof. Ligands and other compositions may be attached on a support material by a non-covalent association or by covalent bonding, using reagents and techniques known in the art. In some embodiments, a ligand is coupled to a chromatography material using a linker.

In one aspect, the disclosure provides an affinity agent (affinity separation matrix) comprised of a library of the disclosure, a ligand or multimer coupled to an insoluble support. Such a support may be one or more particles, such as beads; membranes; filters; capillaries; monoliths; and any other format commonly used in chromatography. In an advantageous embodiment of the affinity separation matrix, the support is comprised of substantially spherical particles, also known as beads. Suitable particle sizes may be in the diameter range of 5-500 μm, such as 10-100 μm, e.g., 20-80 μm. In an alternative embodiment, the support is a membrane. To obtain high adsorption capacities, the support is preferably porous, and ligands may be coupled to the external surfaces as well as to the pore surfaces. In an advantageous embodiment of this aspect, the support is porous.

In another aspect, the disclosure relates to a method of preparing a chromatography affinity agent, which method comprises providing ligands as described above, and coupling the ligands to a support. Coupling may be carried out via a nitrogen or sulfur atom of the ligand for example. The ligands may be coupled to the support directly or indirectly via a spacer element to provide an appropriate distance between the support surface and the ligand. Methods for immobilization of protein ligands to porous or non-porous surfaces are well known in this field.

### Production of Ligands

The production of a ligand, useful in practicing the provided methods, may be carried out using a variety of standard techniques for chemical synthesis, semi-synthetic methods, and recombinant DNA methodologies known in the art. Also provided are methods for producing a ligand, individually or as part of multi-domain fusion protein, as soluble agents and cell associated proteins. In some embodiments, the overall production scheme for a ligand comprises obtaining a reference protein scaffold and identifying a plurality of residues within the scaffold for modification. Depending on the embodiment, a reference scaffold may comprise a protein structure with one or more alpha-helical regions, or other tertiary structure. Once identified, any of a plurality of residues can be modified, for example by substitution of one or more amino acids. In some embodiments, one or more conservative substitutions are made. In some embodiments, one or more non-conservative substitutions are made. In some embodiments a natural amino acid (e.g., one of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine) is substituted into a reference scaffold at targeted positions for modification. In some embodiments, modifications do not include substituting in either a cysteine or a proline. After modifications have been made at identified positions desired in a particular embodiment, the resulting modified polypeptides (e.g., candidate ligands) can be recombinantly expressed, for example in a plasmid, bacteria, phage, or other vector (e.g. to increase the number of each of the modified polypeptides). The modified polypeptides can then be purified and screened to identify those modified polypeptides that have specific binding to a particular target of interest. Modified polypeptides may show enhanced binding specificity for a target of interest as compared to a reference scaffold or may exhibit little or no binding to a given target of interest (or to a non-target protein). In some embodiments, depending on the target of interest, the reference scaffold may show some interaction (e.g., nonspecific interaction) with a target of interest, while certain modified polypeptides will exhibit at least about two-fold, at least about five-fold, at least about 10-fold, at least about 20-fold, at least about 50 fold, or at least about 100-fold (or more) increased binding specificity for the target of interest. Additional details regarding production, selection, and isolation of ligand are provided in more detail below.

### Recombinant Expression of Ligands

In some embodiments, a ligand such as a ligand fusion protein is “recombinantly produced,” (i.e., produced using recombinant DNA technology). Exemplary recombinant methods available for synthesizing ligand fusion proteins, include, but are not limited to polymerase chain reaction (PCR) based synthesis, concatemerization, seamless cloning, and recursive directional ligation (RDL) (see, e.g., Meyer et al., Biomacromolecules 3:357-367 (2002), Kurihara et al., Biotechnol. Lett. 27:665-670 (2005), Haider et al., Mol. Pharm. 2:139-150 (2005); and McMillan et al., Macromolecules 32(11):3643-3646 (1999).

In another aspect, nucleic acids comprising a polynucleotide sequence encoding a ligand or multimer according to the embodiments disclosed above are also provided. Thus, the disclosure encompasses all forms of the present nucleic acid sequence such as RNA and DNA encoding the polypeptide (ligand). The disclosure provides vectors, such as plasmids, which in addition to the coding sequence comprise the required signal sequences for expression of the polypeptide or multimer according the disclosure. Such polynucleotides optionally further comprise one or more expression control elements. For example, a polynucleotide can comprise one or more promoters or transcriptional enhancers, ribosomal binding sites, transcription termination signals, and polyadenylation signals, as expression control elements. A polynucleotide can be inserted within any suitable vector, which can be contained within any suitable host cell for expression.

The expression of nucleic acids encoding ligands is typically achieved by operably linking a nucleic acid encoding the ligand to a promoter in an expression vector. Typical expression vectors contain transcription and translation terminators, initiation sequences, and promoters useful for regulation of the expression of the desired nucleic acid sequence. Exemplary promoters useful for expression in E. coli include, for example, the T7 promoter.

Methods known in the art can be used to construct expression vectors containing the nucleic acid sequence encoding a ligand along with appropriate transcriptional/translational control signals. These methods include, but are not limited to in vitro recombinant DNA techniques, synthetic techniques and in vivo recombination/genetic recombination. The expression of the polynucleotide can be performed in any suitable expression host known in the art including, but not limited to, bacterial cells, yeast cells, insect cells, plant cells or mammalian cells. In some embodiments, a nucleic acid sequence encoding a ligand is operably linked to a suitable promoter sequence such that the nucleic acid sequence is transcribed and/or translated into ligand in a host.

A variety of host-expression vector systems can be utilized to express a nucleic acid encoding a ligand. Vectors containing the nucleic acids encoding a ligand (e.g., individual ligand subunits or ligand fusions) or portions or fragments thereof, include plasmid vectors, a single and double-stranded phage vectors, as well as single and double-stranded RNA or DNA viral vectors. Phage and viral vectors may also be introduced into host cells in the form of packaged or encapsulated virus using known techniques for infection and transduction. Moreover, viral vectors may be replication competent or alternatively, replication defective. Alternatively, cell-free translation systems may also be used to produce the protein using RNAs derived from the DNA expression constructs (see, e.g., WO86/05807 and WO89/01036; and U.S. Pat. No. 5,122,464).

Generally, any type of cell or cultured cell line can be used to express a ligand provided herein. In some embodiments a background cell line used to generate an engineered host cell is a phage, a bacterial cell, a yeast cell or a mammalian cell. A variety of host-expression vector systems may be used to express the coding sequence a ligand fusion protein. Mammalian cells can be used as host cell systems transfected with recombinant plasmid DNA or cosmid DNA expression vectors containing the coding sequence of the target of interest and the coding sequence of the fusion polypeptide. The cells can be primary isolates from organisms, cultures, or cell lines of transformed or transgenic nature.

Suitable host cells include but are not limited to microorganisms such as, bacteria (e.g., E. coli, B. subtilis) transformed with recombinant bacteriophage DNA, plasmid DNA or cosmid DNA expression vectors containing ligand coding sequences; yeast (e.g., Saccharomyces, Pichia) transformed with recombinant yeast expression vectors containing ligand coding sequences; insect cell systems infected with recombinant virus expression vectors (e.g., Baculovirus) containing ligand coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing ligand coding sequences.

Prokaryotes useful as host cells in producing a ligand include gram negative or gram positive organisms such as, E. coli and B. subtilis. Expression vectors for use in prokaryotic host cells generally contain one or more phenotypic selectable marker genes (e.g., genes encoding proteins that confer antibiotic resistance or that supply an autotrophic requirement). Examples of useful prokaryotic host expression vectors include the pKK223-3 (Pharmacia, Uppsala, Sweden), pGEMl (Promega, Wis., USA), pET (Novagen, Wis., USA) and pRSET (Invitrogen, Calif., USA) series of vectors (see, e.g., Studier, J. Mol. Biol. 219:37 (1991) and Schoepfer, Gene 124:83 (1993)). Exemplary promoter sequences frequently used in prokaryotic host cell expression vectors include T7, (Rosenberg et al., Gene 56:125-135 (1987)), beta-lactamase (penicillinase), lactose promoter system (Chang et al., Nature 275:615 (1978)); and Goeddel et al., Nature 281:544 (1979)), tryptophan (trp) promoter system (Goeddel et al., Nucl. Acids Res. 8:4057, (1980)), and tac promoter (Sambrook et al., 1990, Molecular Cloning, A Laboratory Manual, 2d Ed., Cold Spring Harbor Laboratory, Cold Spring Harbor, N.Y.).

In some embodiments, a eukaryotic host cell system is used, including yeast cells transformed with recombinant yeast expression vectors containing the coding sequence of a ligand. Exemplary yeast that can be used to produce compositions of the disclosure, include yeast from the genus Saccharomyces, Pichia, Actinomycetes and Kluyveromyces. Yeast vectors typically contain an origin of replication sequence from a 2mu yeast plasmid, an autonomously replicating sequence (ARS), a promoter region, sequences for polyadenylation, sequences for transcription termination, and a selectable marker gene. Examples of promoter sequences in yeast expression constructs include, promoters from metallothionein, 3-phosphoglycerate kinase (Hitzeman, J. Biol. Chem. 255:2073 (1980)) and other glycolytic enzymes, such as, enolase, glyceraldehyde-3-phosphate dehydrogenase, hexokinase, pyruvate decarboxylase, phosphofructokinase, glucose-6-phosphate isomerase, 3-phospho glycerate mutase, pyruvate kinase, triosephosphate isomerase, phosphoglucose isomerase, and glucokinase. Additional suitable vectors and promoters for use in yeast expression as well as yeast transformation protocols are known in the art. See, e.g., Fleer, Gene 107:285-195 (1991) and Hinnen, PNAS 75:1929 (1978).

Insect and plant host cell culture systems are also useful for producing the compositions of the disclosure. Such host cell systems include for example, insect cell systems infected with recombinant virus expression vectors (e.g., baculovirus) containing the coding sequence of a ligand; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the coding sequence of a ligand, including, but not limited to, the expression systems taught in U.S. Pat. No. 6,815,184; U.S. Publ. Nos. 60/365,769, and 60/368,047; and WO2004/057002, WO2004/024927, and WO2003/078614.

In some embodiments, host cell systems may be used, including animal cell systems infected with recombinant virus expression vectors (e.g., adenoviruses, retroviruses, adeno-associated viruses, herpes viruses, lentiviruses) including cell lines engineered to contain multiple copies of the DNA encoding a ligand either stably amplified (CHO/dhfr) or unstably amplified in double-minute chromosomes (e.g., murine cell lines). In some embodiments, a vector comprising a polynucleotide(s) encoding a ligand is polycistronic. Exemplary mammalian cells useful for producing these compositions include 293 cells (e.g., 293T and 293F), CHO cells, BHK cells, NS0 cells, SP2/0 cells, YO myeloma cells, P3X63 mouse myeloma cells, PER cells, PER.C6 (Crucell, Netherlands) cells VERY, Hela cells, COS cells, MDCK cells, 3T3 cells, W138 cells, BT483 cells, Hs578T cells, HTB2 cells, BT20 cells, T47D cells, CRL7030 cells, HsS78Bst cells, hybridoma cells, and other mammalian cells. Additional exemplary mammalian host cells that are useful in practicing the disclosure include but are not limited, to T cells. Exemplary expression systems and selection methods are known in the art and, including those described in the following references and references cited therein: Borth et al., Biotechnol. Bioen. 71(4):266-73 (2000), in Werner et al., Arzneimittelforschung/Drug Res. 48(8):870-80 (1998), Andersen et al., Curr. Op. Biotechnol. 13:117-123 (2002), Chadd et al., Curr. Op, Biotechnol. 12:188-194 (2001), and Giddings, Curr. Op. Biotechnol. 12:450-454 (2001). Additional examples of expression systems and selection methods are described in Logan et al., PNAS 81:355-359 (1984), Birtner et al. Methods Enzymol. 153:51-544 (1987)). Transcriptional and translational control sequences for mammalian host cell expression vectors are frequently derived from viral genomes. Commonly used promoter sequences and enhancer sequences in mammalian expression vectors include, sequences derived from Polyoma virus, Adenovirus 2, Simian Virus 40 (SV40), and human cytomegalovirus (CMV). Exemplary commercially available expression vectors for use in mammalian host cells include pCEP4 (Invitrogen) and pcDNA3 (Invitrogen).

Physical methods for introducing a nucleic acid into a host cell (e.g., a mammalian host cell) include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York).

Biological methods for introducing a polynucleotide of interest into a host cell include the use of DNA and RNA vectors. Viral vectors, and especially retroviral vectors, have become the most widely used method for inserting genes into mammalian (e.g., human) cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Methods for introducing a DNA and RNA polynucleotides of interest into a host cell include electroporation of cells, in which an electrical field is applied to cells in order to increase the permeability of the cell membrane, allowing chemicals, drugs, or polynucleotides to be introduced into the cell. Ligand containing DNA or RNA constructs may be introduced into mammalian or prokaryotic cells using electroporation.

In some embodiments, electroporation of cells results in the expression of a ligand-CAR on the surface of T cells, NK cells, NKT cells. Such expression may be transient or stable over the life of the cell. Electroporation may be accomplished with methods known in the art including MaxCyte GT® and STX ° Transfection Systems (MaxCyte, Gaithersburg, MD, USA).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). In the case where a non-viral delivery system is utilized, an exemplary delivery vehicle is a liposome. The use of lipid formulations is contemplated for the introduction of the nucleic acids into a host cell (in vitro, ex vivo or in vivo). In some embodiments, the nucleic acid is associated with a lipid. A nucleic acid associated with a lipid can be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they can be present in a bilayer structure, as micelles, or with a “collapsed” structure. They can also simply be interspersed in a solution, possibly forming aggregates that are not uniform in size or shape. Lipids are fatty substances which can be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristoyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristoyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform may be used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., Glycobiology 5:505-510 (1991)). However, compositions that have different structures in solution than the normal vesicular structure are also encompassed. For example, the lipids can assume a micellar structure or merely exist as non-uniform aggregates of lipid molecules. Also contemplated are lipofectamine-nucleic acid complexes.

Regardless of the method used to introduce exogenous nucleic acids into a host cell, the presence of the recombinant nucleic acid sequence in the host cell can routinely be confirmed through a variety of assays known in the art. Such assays include, for example, “molecular biological” assays known in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

Reporter genes are used for identifying potentially transfected cells and for evaluating the functionality of regulatory sequences. In general, a reporter gene is a gene that is not present in or expressed by the recipient organism, tissue, or cell and that encodes a polypeptide whose expression is manifested by some easily detectable property, e.g., enzymatic activity. Expression of the reporter gene is assayed at a suitable time after the DNA has been introduced into the recipient cells. Suitable reporter genes include, but are not limited to, genes encoding luciferase, beta-galactosidase, chloramphenicol acetyl transferase, secreted alkaline phosphatase, or the green fluorescent protein gene (e.g., Ui-Tei et al., FEBS Lett. 479:79-82 (2000)). Suitable expression systems are known in the art and can be prepared using known techniques or obtained commercially. In general, the construct with the minimal 5′ flanking region showing the highest level of expression of reporter gene is identified as the promoter. Such promoter regions can routinely be linked to a reporter gene and used to evaluate agents for the ability to modulate promoter-driven transcription.

A number of selection systems can be used in mammalian host-vector expression systems, including, but not limited to, the herpes simplex virus thymidine kinase, hypoxanthine-guanine phosphoribosyltransferase and adenine phosphoribosyltransferase (Lowy et al., Cell 22:817 (1980)) genes. Additionally, antimetabolite resistance can be used as the basis of selection for e.g., dhfr, gpt, neo, hygro, trpB, hisD, ODC (ornithine decarboxylase), and the glutamine synthase system.

In some embodiments, the initiator N-terminal methionine is included at the NH-terminus of the ligand or the ligands of a library of the disclosure. In many instances the ligand is isolated without the N-terminal methionine residue, which is presumed to be cleaved during expression. In many instances a mixture is obtained with only a proportion of the purified ligand contains the N-terminal methionine. It is obvious to those skilled in the art that the presence or absence of the N-terminal methionine does not affect the functionality of the libraries, ligands and affinity agents described herein.

### Ligand Purification

Once a ligand or a ligand fusion protein has been produced by recombinant expression, it can be purified by methods known in the art for purification of a recombinant protein, for example, by chromatography (e.g., ion exchange, affinity, and sizing column chromatography), centrifugation, differential solubility, or by any other standard technique for the purification of proteins. In some embodiments, a ligand is optionally fused to heterologous polypeptide sequences specifically disclosed herein or otherwise known in the art to facilitate purification. In some embodiments, ligands (e.g., antibodies and other affinity matrices) for ligand affinity columns for affinity purification and that optionally, the ligand or other components of the ligand fusion composition that are bound by these ligands are removed from the composition prior to final preparation of the ligand using techniques known in the art.

### Chemical Synthesis of Ligand

In addition to recombinant methods, ligand production may also be carried out using organic chemical synthesis of the desired polypeptide using a variety of liquid and solid phase chemical processes known in the art. Various automatic synthesizers are commercially available and can be used in accordance with known protocols. See, for example, Tam et al., J. Am. Chem. Soc., 105:6442 (1983); Merrifield, Science, 232:341-347 (1986); Barany and Merrifield, The Peptides, Gross and Meienhofer, eds, Academic Press, New York, 1-284; Barany et al., Int. J. Pep. Protein Res., 30:705 739 (1987); Kelley et al. in Genetic Engineering Principles and Methods, Setlow, J. K., ed. Plenum Press, N Y. 1990, vol. 12, pp. 1-19; Stewart et al., Solid-Phase Peptide Synthesis, W.H. Freeman Co., San Francisco, 1989. One advantage of these methodologies is that they allow for the incorporation of non-natural amino acid residues into the sequence of the ligand.

The ligand that are used in the methods of the present disclosure may be modified during or after synthesis or translation, e.g., by glycosylation, acetylation, benzylation, phosphorylation, amidation, pegylation, formylation, derivatization by known protecting/blocking groups, proteolytic cleavage, linkage to an antibody molecule, hydroxylation, iodination, methylation, myristoylation, oxidation, pegylation, proteolytic processing, phosphorylation, prenylation, racemization, selenoylation, sulfation, ubiquitination, etc. (See, e.g., Creighton, Proteins: Structures and Molecular Properties, 2d Ed. (W.H. Freeman and Co., N.Y., 1992); Postranslational Covalent Modification of Proteins, Johnson, ed. (Academic Press, New York, 1983), pp. 1-12; Seifter, Meth. Enzymol., 182:626-646 (1990); Rattan, Ann. NY Acad. Sci., 663:48-62 (1992).) In some embodiments, the peptides are acetylated at the N-terminus and/or amidated at the C-terminus.

Any of numerous chemical modifications may be carried out by known techniques, including, but not limited to acetylation, formylation, etc. Additionally, derivatives may contain one or more non-classical amino acids.

In some embodiments cyclization, or macrocyclization of the peptide backbone is achieved by sidechain to sidechain linkage formation. Methods for achieving this are well known in the art and may involve natural as well as unnatural amino acids. Approaches includes disulfide formation, lanthionine formation or thiol alkylations (e.g. Michael addition), amidation between amino and carboxylate sidechains, click chemistry (e.g. azide-alkyne condensation), peptide stapling, ring closing metathesis and the use of enzymes.

### Affinity Agents for Purification

In purification based on affinity chromatography, a target of interest (e.g., protein or molecule) is selectively isolated according to its ability to specifically and reversibly bind to a ligand that has typically been covalently coupled to a chromatographic matrix. In some embodiments, ligands identified from the libraries of the disclosure can be used as reagents for affinity purification of targets of interest from either recombinant sources or natural sources such as biological samples (e.g., serum).

In some embodiments, a ligand that specifically binds a target of interest is immobilized on beads and then used to affinity purify the target.

Methods of covalently coupling proteins to a surface are known by those of skill in the art, and peptide tags that can be used to attach ligand to a solid surface are known to those of skill in the art. Further, ligand can be attached (i.e., coupled, linked, or adhered) to a solid surface using any reagents or techniques known in the art. In some embodiments, a solid support comprises beads, glass, slides, chips and/or gelatin. Thus, a series of ligands can be used to make an array on a solid surface using techniques known in the art. For example, U.S. Publ. No. 2004/0009530 discloses methods for preparing arrays.

In some embodiments, a ligand derived from a library of the disclosure is used to isolate its cognate target of interest by affinity chromatography. In some embodiments, such a ligand is immobilized on a solid support. The ligand can be immobilized on the solid support using techniques and reagents described herein or otherwise known in the art. Suitable solid supports are described herein or otherwise known in the art and in specific embodiments are suitable for packing a chromatography column. The affinity agent can be packed in columns of various sizes and operated at various linear velocities or the immobilized affinity ligand can be loaded or contacted with a solution under conditions favorable to form a complex between the ligand and the target of interest. Non-binding materials can be washed away. Suitable wash conditions can readily be determined by one of skill in the art. Examples of suitable wash conditions are described in Shukla and Hinckley, Biotechnol Prog. 2008 September-October; 24(5):1115-21. doi: 10.1002/btpr.50.

In some embodiments, chromatography is carried out by mixing a solution containing the target of interest and the ligand, then isolating complexes of the target of interest and ligand. For example, a ligand is immobilized on a solid support such as beads, then separated from a solution along with the target of interest by filtration. In some embodiments, a ligand is a fusion protein that contains a peptide tag, such as a poly-His tail or streptavidin binding region, which can be used to isolate the ligand after complexes have formed using an immobilized metal affinity chromatographic resin or streptavidin-coated substrate. Once separated, the target of interest can be released from the ligand under elution conditions and recovered in a purified form.

Elution of a target of interest can be achieved by techniques generally known in the art, including by lowering pH and increasing salt concentrations or otherwise altering salt conditions For example, elution of viral particles is generally achieved by lowering the pH, e.g., 2.0-3.0, although higher pH may be used. Optimal conditions for elution of AAV8 and variants and mutants thereof can be readily determined by those of skill in this field.

The affinity agents of the disclosure are alkali-tolerant, enabling the use of NaOH up to concentrations of 0.5 M for cleaning. In certain embodiments, a CIP) regimen of 0.5 M NaOH exposure for up to 30 to 60 minutes per cycle, for example, ensures consistent chromatographic performance for several cycles, e.g., 15-30 cycles, including up to 70%-90% of the initial AAV8 binding capacity and low residual DNA and HCP levels, as well as substantially no change in flow capacity.

While some embodiments in the disclosure have been described by way of illustration, it will be apparent that the invention(s) herein can be put into practice with many modifications, variations and adaptations, and with the use of numerous equivalents or alternative solutions that are within the scope of persons skilled in the art, without departing from the spirit of the invention or exceeding the scope of the claims.

All publications, patents, and patent applications are herein incorporated by reference in their entirety to the same extent as if each individual publication, patent or patent application was specifically and individually indicated to be incorporated by reference in its entirety.

### EXAMPLES

### Example 1: Library Construction and Screening

Phage display libraries were designed based on face variant positions in helices 2 and 3 (see FIGS. 2 and 3) of the Z domain using the scaffold

[A]-X₁QRRX₂FIX₃X₄LRX₅DPSX₆SAX₇LLAX₈AX₉X₁₀X₁₁NDX₁₂-[B].

The scaffolds of the phage libraries were constructed using trimer codon phosphoramidite mutagenesis.

For one library, [A] is VDAKFDKELEEARAEIERLPNLTE; each of the X positions other than X₉ is any amino acid; and X₉ is A, R or K; provided that at least two of the following criteria are met: X₂ is not alanine (A), X₄ is not serine (S), X₅ is not aspartic acid (D), X₆ is not glutamine or serine (Q or S), X₈ is not glutamic acid (E), X₁₀ is not lysine (L) and X₁₁ is not leucine (L).

and at least two of the following are true, that met.

For another library, [A] is VDAKFDKELEEARAEIERLPNLTE, each of the X positions other than X₉ and X₁₂ is any amino acid, and each of X₉ and X₁₂ are A, R or K; provided that at least two of the following criteria are met: X₂ is not alanine (A), X₄ is not serine (S), X₅ is not aspartic acid (D), X₆ is not glutamine or serine (Q or S), X₈ is not glutamic acid (E), X₁₀ is not lysine (L) and X₁₁ is not leucine (L).

Phage library panning is performed as generally described in (Griffiths et al. 1994, EMBO J., 13:3245-3260). Multiple rounds of panning are performed as needed against a target of interest, including, for example, AAV8 capsids.

Individual phage clones can be tested for binding to a target of interest in a phage ELISA. Briefly, 1×10¹² phage are incubated in 96-well plates coated at 1 μg/mL with a target of interest and a negative control. After incubating for one hour at room temperature, unbound particles are removed by washing the wells three times in PBS-0.1% Tween-20. Bound bacteriophage are detected using a specific anti-M13 antibody, isolated and sequenced to identify affinity ligands that bind the target of interest. After identification, the affinity ligands can be prepared as peptides or produced recombinantly.

Peptides are synthesized by standard Fmoc solid phase peptide synthesis techniques and purified by preparative reverse phase HPLC. The purity of peptides is assessed by RP-UPLC with both UV and quadrupole time-of-flight mass spectrometric detection.

Recombinant affinity ligands are expressed in E. coli or Pichia pastoris using standard techniques. Ligands can be purified using multi-column chromatography. For His-tagged ligands, immobilized metal affinity chromatography (IMAC) is used as the primary capture step. Biotinylated ligands are generated with the AviTag™ system (Avidity, Aurora, CO). Non-biotinylated ligands bearing the AviTag™ sequence are prepared by omitting exogenous biotin. The purity and identity of recombinant protein ligands is assessed by a combination of SDS-PAGE, RP UPLC, quadrupole time-of-flight mass spectrometry and size exclusion chromatography (SEC); Sephadex S75, Cytiva, Marlborough, MA). In many instances the ligand is isolated without the N-terminal methionine residue, which is presumed to be cleaved during expression.

### Example 2. Exemplary AAV8 Affinity Ligand

This example demonstrates the binding of biotinylated ligands to AAV8 capsids based on an affinity ligand obtained from the library described in Example 1 using biolayer interferometry (ForteBio, Menlo Park, CA). Biotinylated ligands, were immobilized on sensors and incubated with solutions containing 5×10¹¹ particles/mL in 100 mM sodium phosphate, 100 mM sodium chloride, 0.01% (w/v) bovine serum albumin and 0.1% (v/v) Triton X-100, pH 7.0. A blank sensor and a non-binding ligand were included as controls. The association phase showed the initial linear increase in response that it is typical for AAV. As the sensor became saturated, the sensorgram showed greater curvature (FIG. 4, ligand 1). Responses were measured after 4000 seconds incubation time and shown in Table 2 below for exemplary AAV8 ligands.

TABLE 2

| Ligand | Response |
|---|---|
| 1 | 7.31 |
| 2 | 4.47 |
| 3 | 6.48 |
| 4 | 5.99 |
| 5 | 6.19 |
| 6 | 6.29 |
| 7 | 5.56 |
| 8 | 6.00 |
| 9 | 6.16 |
| 10 | 6.21 |
| 11 | 5.91 |
| 12 | 6.23 |
| 13 | 4.94 |
| 14 | 4.47 |
| 15 | 6.06 |
| 16 | 5.85 |
| 17 | 6.18 |
| 18 | 5.79 |
| 19 | 4.95 |
| 20 | 5.60 |
| Blank sensor | 0.05 |
| Non-binding ligand | 0.03 |

### Example 3. Alkaline Stability of Library Affinity Ligands

This example demonstrates the sodium hydroxide stability of the affinity ligands. Ligands were incubated in 0.5 M NaOH for 16 hours and then neutralized. The binding of the NaOH treated ligands was measured as described in Example 2 and compared to untreated ligand. The binding retained was calculated according to the following formula:

% binding retained=(measured response after NaOH treatment)÷(measured response of untreated)×100

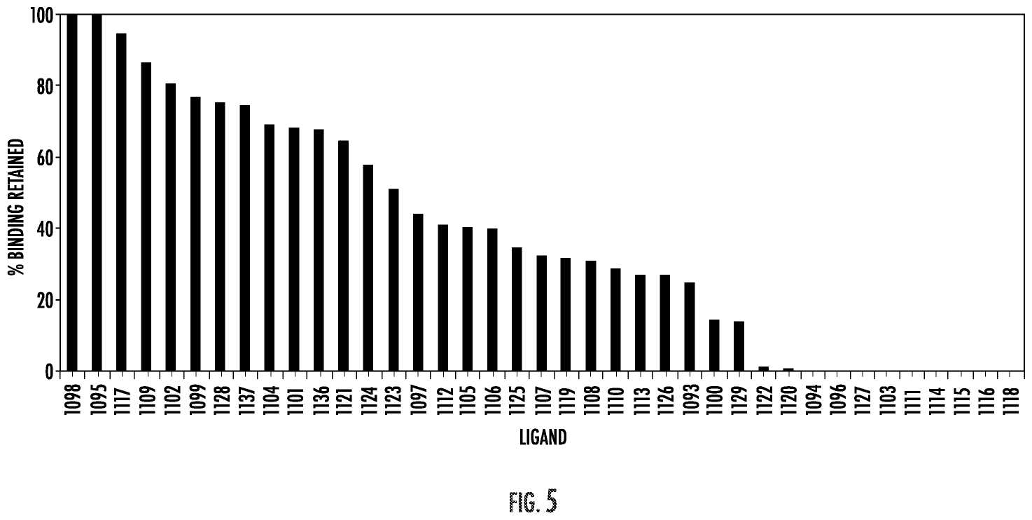

The data demonstrates that the affinity ligands of the library can possess high alkaline stability (FIG. 5).

### Example 4. Construction of Exemplary Affinity Resins

This example demonstrates the production and characterization of affinity agents comprising ligands identified and described herein. Affinity resins were prepared by conjugating ligands to agarose beads. RAPID RUN 6% Agarose beads (ABT, Madrid, Spain) and Praesto® Jetted A50 beads (Purolite, King of Prussia, PA) were activated with disuccinimidyl carbonate and coupled with peptide ligands at ligand densities 1-8 mg/mL resin. The actual ligand density for all resins was measured using a subtractive RP-HPLC method according to the following formula:

Actual Ligand Density=(Amount of ligand in feed−Amount of ligand in effluent)/volume of resin.

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims and list of embodiments disclosed herein. Although specific terms are employed herein, they are used in a generic and descriptive sense only and not for purposes of limitation.

Any methods disclosed herein need not be performed in the order recited. The methods disclosed herein include certain actions taken by a practitioner; however, they can also include any third-party instruction of those actions, either expressly or by implication.

1. A nucleic acid library whose members encode an affinity ligand comprising an amino acid sequence represented by the formula, from N-terminus to C-terminus,
(SEQ ID NO. 1)
[A]-X₁QRRX₂FIX₃X₄LRX₅DPS-[X₆]n-SAX₇LLAX₈AX₉X₁₀X₁₁ND
X₁₂QAPX₁₃-[B],
wherein (a) [A] comprises an α-helix-forming peptide domain;
(b) each of X₁, X₂, X₃, X₄, X₅, X₆, X₇, X₈, X₁₀, X₁₁, and X₁₂ is independently any amino acid;
(c) n represents the number of X₆ residues present and is an integer from one to ten,
(d) each of X₉ and X₁₃ is independently A, K or R; and
(e) [B] is absent, is VD, or is a peptide domain comprising an amino acid sequence of
(SEQ ID NO.9)
VDGQAGQGGGSGLNDIFEAQKIEWHEHHHHHH,
(SEQ ID NO. 10)
GQAGQGGGSGLNDIFEAQKIEWHEHHHHHH,
(SEQ ID NO. 11)
VDGLNDIFEAQKIEWHEHHHHHH
or
(SEQ ID NO. 12)
GLNDIFEAQKIEWHEHHHHHH

2. The nucleic acid library of claim 1, wherein said α-helix-forming peptide domain comprises an alkali-stable helix 1 of a staphylococcal protein A (SPA) domain found at residues 5-19 of any one of an SPA Z-domain, A-domain, B-domain, C-domain, D-domain and E-domain, and preferably a Z-domain.

3. The nucleic acid library of claim 1, wherein [A] comprises a peptide having an amino acid sequence of
(SEQ ID NO. 2)
VDAKFDKELEEARAEIERLPNLTE,
(SEQ ID NO. 3)
VDAKFDKELEEARAKIERLPNLTE,
(SEQ ID NO. 4)
VDAKFDKELEEVRAEIERLPNLTE,
(SEQ ID NO. 5)
VDAKFEKELEEARAEIERLPNLTE,
(SEQ ID NO. 6)
VDAKFDKELEEIRAEIERLPNLTE
or
(SEQ ID NO. 7)
VDAKFDKELEEARAEIERLPALTE.

4. The nucleic acid library of claim 3, wherein the N-terminus of [A] is preceded by M or MAQGT (SEQ ID NO. 8).

5. The nucleic acid library of claim 1, which comprises a peptide tag, optionally, wherein said tag is hemagglutinin, c-myc, a herpes simplex virus glycoprotein D, T7, GST, GFP, MBP, a strep-tag, a His-tag, a Myc-tags, a TAP-tag or a FLAG tag.

6. The nucleic acid library of claim 1, wherein said ligand comprises a C-terminal lysine or cysteine.

7. The nucleic acid library of claim 1, wherein [A] is VDAKFDKELEEARAEIERLPNLTE (SEQ ID NO. 2), n is one, and X₁₃ is K.

8. The nucleic acid library of claim 1, wherein said library comprises any one of SEQ ID NOS. 13-18.

9. The nucleic acid library of claim 1, wherein said library is a phage display library, a yeast display library, an RNA display library or a DNA display library.

10. A method of identifying a polypeptide that interacts selectively with a target molecule of interest which comprises:
a) exposing a target molecule of interest to polypeptides produced by expression of a nucleic acid library of claim 1; and
b) separating polypeptides that selectively interact from those that do not selectively interact with the target molecule.

11. The method of claim 10, wherein the target molecule of interest is expressed on the surface of a phage, bacterium or cell, or is attached to, tethered to or otherwise associated with a solid support.

12. A method of screening a library for a polypeptide that specifically binds with high affinity to a target molecule of interest, the library comprising a plurality of polypeptides produced by expression of a nucleic acid library of claim 1, which comprises:
(a) incubating a sample of the library with a concentration of a target molecule under conditions suitable for specific binding of the polypeptides to the molecule;
(b) incubating a second sample of the library under the same conditions but without target molecule;
(c) contacting each of the first and second samples with immobilized target molecule under conditions suitable for binding of the polypeptide to the immobilized target molecule;
(d) detecting the polypeptide bound to immobilized target molecule for each sample; and
(e) determining the affinity of the polypeptide for the target molecule by calculating the ratio of the amounts of bound polypeptide from the first sample over the amount bound polypeptide from the second sample.

13. A method of identifying one or more affinity ligands that specifically binds with a target molecule of interest which comprises:
(a) contacting said target molecule with a phage display library of claim 9;
(b) separating phage that specifically bind with said target molecule from those that do not selectively interact with said target molecule to produce an enriched phage library;
(c) repeating steps a) and b) with said enriched phage library to produce a further enriched phage library;
(d) repeating step c) until said further enriched phage library is enriched from at least about 10- to about 10⁶-fold or more relative to the original phage library; and
(e) plating said further enriched phage library and isolating and characterizing individual clones therefrom and thereby identifying one or more affinity ligands that specifically bind to the target molecule of interest.

14. The method of claim 9, wherein said target molecule or said phage display library is bound to or attached to a solid support.

15. The method of claim 13, wherein said target molecule is an adeno-associated virus (AAV) or AAV capsid.

16. The method of claim 15 wherein said AAV is AAV8 or a AAV8 serotype variant.

17. A composition comprising a plurality of synthetic or recombinant polypeptides, each polypeptide comprising an affinity ligand of claim 1.

18. A method of identifying a polypeptide that binds specifically to a target molecule of interest which comprises:
(a) exposing a target molecule of interest to a composition of claim 17;
(b) separating polypeptides that specifically bind to said target molecule from those that do not selectively bind the target molecule; and
(c) identifying one or more polypeptides bound by said target molecule.
